(19) United States
(12) Patent Application Publication
(10) Pub. No.: US 2022/0152297 A1
(43) Pub. Date: May 19, 2022

(54) INTEGRATED INSERTER/APPLICATOR FOR A DRUG DELIVERY SYSTEM PROVIDING MULTIPLE WEAR CONFIGURATIONS

(71) Applicant: Eli Lilly and Company, Indianapolis, IN (US)

(72) Inventors: Michel Gilbert BRUEHWILER, Newton, MA (US); Eileen Mei BUTLER, King of Prussia, PA (US); Mary Teresa CARTER, Boston, MA (US); Daniel Morris HARTMANN, Arlington, MA (US); Ariel Marie ROBERTS, Greenwood, IN (US)

(21) Appl. No.: 17/439,066

(22) PCT Filed: Mar. 20, 2020
(86) PCT No.: PCT/US2020/023825

§ 371 Date: Sep. 14, 2021

Related U.S. Application Data
(60) Provisional application No. 62/822,318, filed on Mar. 22, 2019.

(51) Int. Cl. A61M 5/142; A61M 5/158; A61M 5/162; A61M 5/14

(52) U.S. Cl. CPC A61M 5/14248; A61M 5/158; A61M 5/162; A61M 2005/1587; A61M 2005/14252; A61M 2005/1585; A61M 2005/1586; A61M 5/1413

(57) ABSTRACT

An applicator assembly for use with a dmg infusion system having multiple wear configurations, including a tethered configuration and a patch configuration. The applicator assembly may be a multi-functional device configured to (1) selectively apply an infusion head and/or a holder to a patients skin and (2) insert an injection device of the infusion head into the patients skin. The drug infusion system may be worn interchangeably in the tethered configuration by placing a dmg delivery device in fluid communication with the infusion head via an intermediate tubing set, or in the patch configuration by coupling the drug delivery device to the holder and placing the dmg delivery device in fluid communication with the infusion head without the intermediate tubing set.

### CROSS REFERENCE TO RELATED APPLICATION

This application claims priority to U.S. Provisional Patent Application Ser. No. 62/822,318, filed Mar. 22, 2019, the disclosure of which is hereby expressly incorporated by reference herein in its entirety.

### FIELD OF THE DISCLOSURE

The present disclosure relates to drug infusion systems, more specifically applicator assemblies for use with drug infusion systems having multiple wear configurations, and to methods of using the same.

### BACKGROUND OF THE DISCLOSURE

A drug infusion system includes a drug delivery device (e.g., pump) configured to store and deliver a drug (e.g., insulin) into a patient's skin via an infusion head. Some drug infusion systems may be used in a tethered configuration, wherein the infusion head is adhered to the patient's skin, the drug delivery device is positioned in a remote location apart from the patient's skin, such as a pouch, pocket, or case, and an intermediate tubing set is connected therebetween. Other drug infusion systems may be used in a patch (i.e., wearable) configuration, in which both the infusion head and the drug delivery device are adhered to the patient's skin without an intermediate tubing set. Manufacturers typically supply devices that can be used in only one of these configurations; accordingly, patients typically do not have the option to switch from one configuration to another.

### SUMMARY

The present disclosure provides an applicator assembly for use with a drug infusion system having multiple wear configurations, including a tethered configuration and a patch configuration. The applicator assembly may be a multi-functional device configured to (1) selectively apply an infusion head and/or a holder to a patient's skin and (2) insert an injection device of the infusion head into the patient's skin. The drug infusion system may be worn interchangeably in the tethered configuration by placing a drug delivery device in fluid communication with the infusion head via an intermediate tubing set, or in the patch configuration by coupling the drug delivery device to the holder and placing the drug delivery device in fluid communication with the infusion head without the intermediate tubing set.

According to an embodiment of the present disclosure, an applicator assembly is disclosed that is configured for use with a drug infusion system including a drug delivery device, the drug infusion system having a tethered configuration and a patch configuration. The applicator assembly includes an infusion head having a first adhesive pad and an injection device, a holder having a second adhesive pad, the holder configured to support the drug delivery device, a housing configured to carry the infusion head and the holder, and an actuator coupled to the housing and configured to move the injection device from the housing into a patient's skin.

According to another embodiment of the present disclosure, a drug infusion system is disclosed including a drug delivery device, an infusion head having an injection device, a holder configured to support the drug delivery device, and an applicator assembly including a housing configured to carry the infusion head and the holder, and an actuator coupled to the housing and configured to move the injection device from the housing into a patient's skin. The drug infusion system has a tethered configuration in which the applicator assembly positions the infusion head in adhesive communication with the patient's skin while preventing adhesive communication between the holder and the patient's skin and a patch configuration in which the applicator assembly positions the infusion head and the holder in adhesive communication with the patient's skin.

According to yet another embodiment of the present disclosure, a method is disclosed for using a drug infusion system including the steps of obtaining an applicator assembly comprising a housing, an infusion head having a first adhesive pad and an injection device, and a holder having a second adhesive pad, preparing the applicator assembly by selectively concealing or exposing the second adhesive pad of the holder, applying the applicator assembly to a patient's skin, the holder adhering to the patient's skin during the applying step if the second adhesive pad is exposed during the preparing step, actuating the applicator assembly to adhere the first adhesive pad of the infusion head to the patient's skin and insert the injection device into the patient's skin, and separating the applicator assembly from the patient's skin, the holder separating from the patient's skin during the separating step if the second adhesive pad is concealed during the preparing step.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is an exploded perspective view of an exemplary drug infusion system of the present disclosure, the drug infusion system including a drug delivery device, an optional tubing set, an infusion head, and a holder;

FIG. 2 is a perspective view of the drug delivery device of FIG. 1;

FIG. 3 is a perspective view of the tubing set of FIG. 1;

FIG. 4 is a perspective view of the drug infusion system of FIG. 1 arranged in a tethered configuration with the tubing set;

FIG. 5 is a perspective view of the drug infusion system of FIG. 1 arranged in a patch configuration without the tubing set;

FIG. 6 is a bottom perspective view of a first exemplary applicator assembly of the present disclosure configured for use with the drug infusion system of FIG. 1, the first applicator assembly shown in a preparation state;

FIG. 7 is a cross-sectional view of the first applicator assembly of FIG. 6 shown in an application state;

FIG. 8 is a cross-sectional view of the first applicator assembly of FIG. 6 shown in an insertion state;

FIG. 9 is a cross-sectional view of the first applicator assembly of FIG. 6 shown in a retraction state;

FIG. 10 is a cross-sectional view of the first applicator assembly of FIG. 6 shown in a separation state;

FIG. 11 is a cross-sectional view of the first applicator assembly of FIG. 6 shown in a second preparation state;

FIG. 12 is a cross-sectional view of the first applicator assembly of FIG. 6 shown in a second application state;

FIG. 13 is a cross-sectional view of the first applicator assembly of FIG. 6 shown in a second separation state;

FIG. 14 is an exploded perspective view of a second exemplary applicator assembly of the present disclosure configured for use with the drug infusion system of FIG. 1;

FIG. 15 is a cross-sectional view of the second applicator assembly of FIG. 14 shown in a fully locked, preparation state;

FIG. 16 is a cross-sectional view of the second applicator assembly of FIG. 14 shown in a partially locked, application state;

FIG. 17 is a cross-sectional view of the second applicator assembly of FIG. 14 shown in an unlocked, insertion state; and

FIG. 18 is a cross-sectional view of the second applicator assembly of FIG. 14 shown in a relocked, separation state.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

### 1. Drug Infusion System

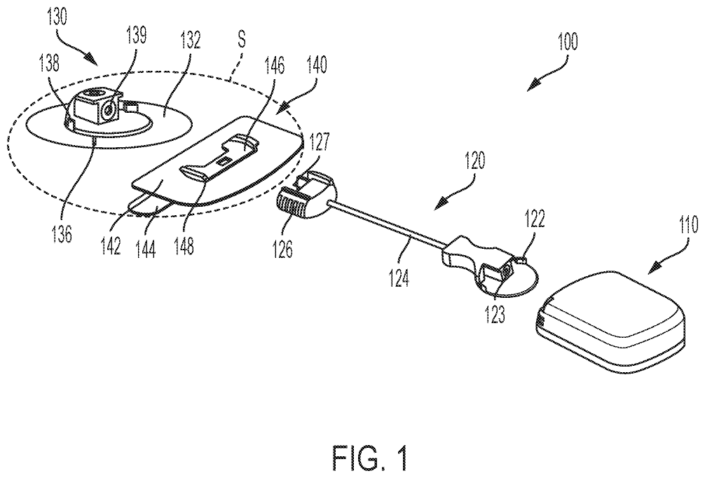

An exemplary drug infusion system 100 of the present disclosure is shown in FIG. 1 for delivering a drug (not shown) into a patient's skin S. The drug infusion system 100 includes a drug delivery device 110, an optional tubing set 120, an infusion head 130 configured for placement on the patient's skin S, and an optional holder 140 also configured for placement against the patient's skin S. Each element of the drug infusion system 100 is described further below.

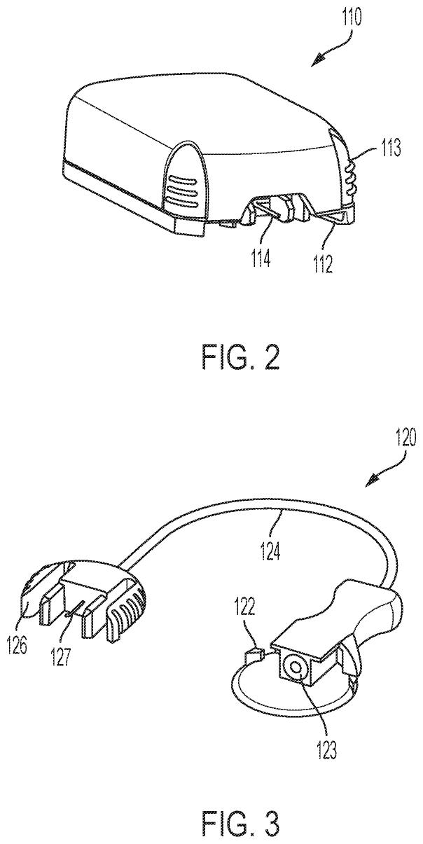

The drug delivery device 110 is configured to store and deliver a drug to the infusion head 130, either indirectly to the infusion head 130 via the tubing set 120 or directly to the infusion head 130 without the tubing set 120. The drug delivery device 110 is also shown in FIG. 2 and includes a connector 112 (e.g., a male buckle) with release latches 113 and a needle port 114 in selective fluid communication with the tubing set 120 or the infusion head 130. The drug delivery device 110 may be a pump, bolus injector, autoinjector, injection pen, or another suitable drug delivery device. The drug delivery device 110 may be controlled via built-in controls (e.g., a touchscreen, buttons) or via wireless controls (e.g., the patient's smartphone). In certain embodiments, a set of multiple drug delivery devices 110 may be provided within the drug infusion system 100, with each drug delivery device 110 containing a different volume of the drug, such as 1 mL to 20 mL or more, for example.

The drug of the drug delivery device 110 may include one or more therapeutic agents including, but not limited to, insulins, insulin analogs (e.g., insulin lispro, insulin glargine), insulin derivatives, GLP-1 receptor agonists (e.g., dulaglutide, liraglutide), glucagon, glucagon analogs, glucagon derivatives, gastric inhibitory polypeptides (GIP), GIP analogs, GIP derivatives, oxyntomodulin analogs, oxyntomodulin derivatives, therapeutic antibodies, and any other therapeutic agents capable of delivery by the drug delivery device 110. The drug may be formulated with one or more excipients.

The optional tubing set 120 is also shown in FIG. 3 and illustratively includes a first connector 122 (e.g., a female buckle) configured to selectively couple to the drug delivery device 110, a first septum port 123 in selective fluid communication with the drug delivery device 110, a flexible line set tubing 124, a second connector 126 (e.g., a male buckle) configured to selectively couple to the infusion head 130, and a second needle port 127 in selective fluid communication with the infusion head 130. The tubing set 120 is configured to convey the drug from the first septum port 123, through the line set tubing 124, and through the second needle port 127.

The infusion head 130 illustratively includes a first adhesive pad 132 configured to adhere the infusion head 130 to the patient's skin S, an injection device in the form of a cannula 136 or a needle configured for insertion into the patient's skin S, and a connector 138 (e.g., a female buckle) configured to selectively couple to the drug delivery device 110 or the tubing set 120. The first adhesive pad 132 may define the outer perimeter of the infusion head 130. The infusion head 130 also includes at least one septum port 139 in selective fluid communication with the needle port 114 of the drug delivery device 110 or the needle port 127 of the tubing set 120. In use, the drug is directed through the septum port 139, through the cannula 136, and into the subcutaneous tissue of the patient's skin S.

The optional holder 140 illustratively includes a second adhesive pad 142 configured to adhere the holder 140 to the patient's skin S, a removable backing 144 configured to selectively cover and expose the second adhesive pad 142, and a connector 146 (e.g., clip) configured to selectively mate with the drug delivery device 110. The connector 146 may have rounded edges 148 to facilitate coupling with the drug delivery device 110. The second adhesive pad 142 may define the outer perimeter of the holder 140.

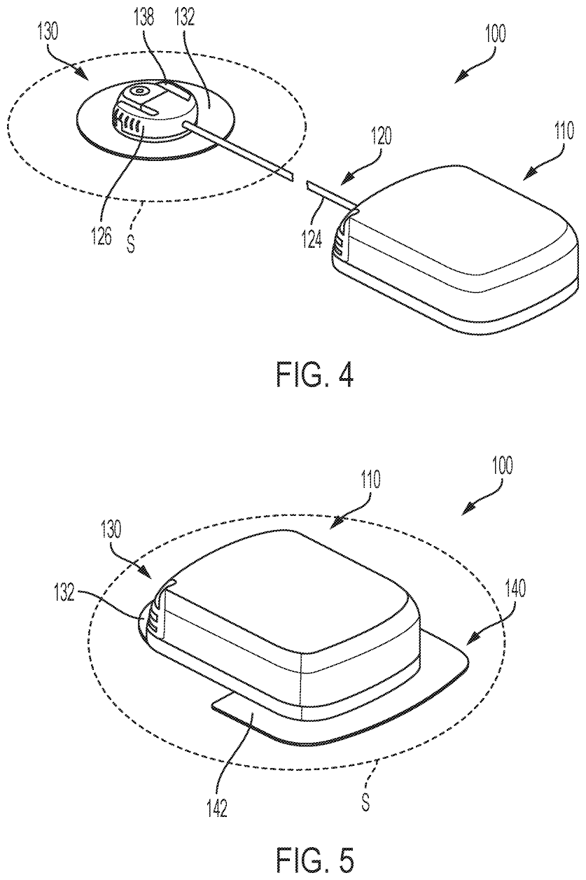

The drug infusion system 100 may be used interchangeably in at least two different wear configurations, including a tethered configuration with the tubing set 120 as shown in FIG. 4 and a patch (i.e., wearable) configuration without the tubing set 120 as shown in FIG. 5. To achieve this interchangeability, the first connector 122 of the tubing set 120 may be the same as or similar to the connector 138 of the infusion head 130, and the second connector 126 of the tubing set 120 may be the same as or similar to the connector 112 of the drug delivery device 110, as shown in FIGS. 1-3. However, these connections may be adjusted based on available space and a desired coupling action (e.g., top-load, side-load, and angled-load). Advantageously, these wear configurations may be adjusted without disturbing the inserted cannula 136 of the infusion head 130. These illustrative wear configurations are described further below, but other wear configurations are also within the scope of the present disclosure.

In the tethered configuration of FIG. 4, the infusion head 130 is adhered to the patient's skin S, while the drug delivery device 110 is positioned in a remote location apart from the patient's skin S, such as a pouch, pocket, or case. The tubing set 120 is positioned in fluid communication between the tethered drug delivery device 110 and the infusion head 130 such that the tethered drug delivery device 110 conveys the drug through the tubing set 120 and then to the infusion head 130 for delivery to the patient. In this arrangement, the connector 112 of the drug delivery device 110 (FIG. 2) mates with the first connector 122 of the tubing set 120 (FIG. 1), and the second connector 126 of the tubing set 120 mates with the connector 138 of the infusion head 130. Also, the needle port 114 of the drug delivery device 110 (FIG. 2) pierces the first septum port 123 of the tubing set 120, and the second needle port 127 of the tubing set 120 pierces the septum port 139 of the infusion head 130 (FIG. 1). Small compression springs (not shown) may be provided around the needle port 114 of the drug delivery device 110 and/or the second needle port 127 of the tubing set 120 to facilitate detachment from the corresponding septum ports 123, 139. The patient may prefer to use the drug infusion system 100 in the tethered configuration when the drug delivery device 110 is relatively large and heavy (e.g., about 3.5 oz or more), which may coincide with the drug delivery device 110 containing a large volume of the drug.

Although not shown in FIG. 4, the holder 140 may be optionally adhered to the patient's skin S in the tethered configuration. In certain embodiments, the holder 140 may be bypassed or ignored in the tethered configuration. In other embodiments, the holder 140 may interact with (e.g., grip) the tubing set 120 to provide strain-relief for the tubing set 120.

In the patch configuration of FIG. 5, both the infusion head 130 and the holder 140 are adhered to the patient's skin S. The drug delivery device 110 is coupled to the holder 140 such that the drug delivery device 110 is supported on the patient's skin S. The drug delivery device 110 is also arranged in direct fluid communication with the infusion head 130 such that the drug delivery device 110 conveys the drug directly to the infusion head 130 for delivery to the patient. In this arrangement, the connector 112 of the drug delivery device 110 (FIG. 2) directly mates with the connector 138 of the infusion head 130. Also, the needle port 114 of the drug delivery device 110 (FIG. 2) directly pierces the septum port 139 of the infusion head 130 (FIG. 1). As noted above, a small compression spring (not shown) may be provided around the needle port 114 of the drug delivery device 110 to facilitate detachment from the corresponding septum port 139. The patient may prefer to use the drug infusion system 100 in the patch configuration when the drug delivery device 110 is relatively small and light (e.g., less than about 3.5 oz, less than about 1.75 oz, etc.), which may coincide with the drug delivery device 110 containing a small volume of the drug.

As noted above, the coupling action of the drug delivery device 110 to the infusion head 130 and the holder 140 in the patch configuration may vary. In a top-load embodiment, the drug delivery device 110 may be coupled to the infusion head 130 and the holder 140 in a direction substantially normal to the skin S. In an angled-load embodiment, the drug delivery device 110 may be coupled to the infusion head 130 and the holder 140 via a tilt-in approach. For example, one end of the drug delivery device 110 may be hooked onto the holder 140, while the other end of the drug delivery device 110 may be pivoted onto the infusion head 130. In a side-load embodiment, the drug delivery device 110 may be coupled to the infusion head 130 and the holder 140 in a direction substantially parallel to the skin S. For example, the drug delivery device 110 may slide over the holder 140 and then lock into position on the infusion head 130. The rounded edges 148 on the connector 146 may facilitate this coupling action by allowing to drug delivery device 110 to pivot relative to the connector 146 during alignment with the infusion head 130.

In the patch configuration, the tubing set 120 of FIG. 3 may be stored. In certain embodiments, the tubing set 120 may be stored in a closed-loop manner with the first connector 122 of the tubing set 120 coupled to the second connector 126 of the tubing set 120. In this arrangement, the second needle port 127 of the tubing set 120 pierces the septum port 123 of the tubing set 120, thereby protecting the second needle port 127 without the need for a separate needle shield, preventing unwanted contact with the second needle port 127, and sealing any drug product that remains inside the tubing set 120. As noted above, a small compression spring (not shown) may be provided around the second needle port 127 of the tubing set 120 to facilitate detachment from the corresponding septum port 123.

### 2. First Applicator Assembly

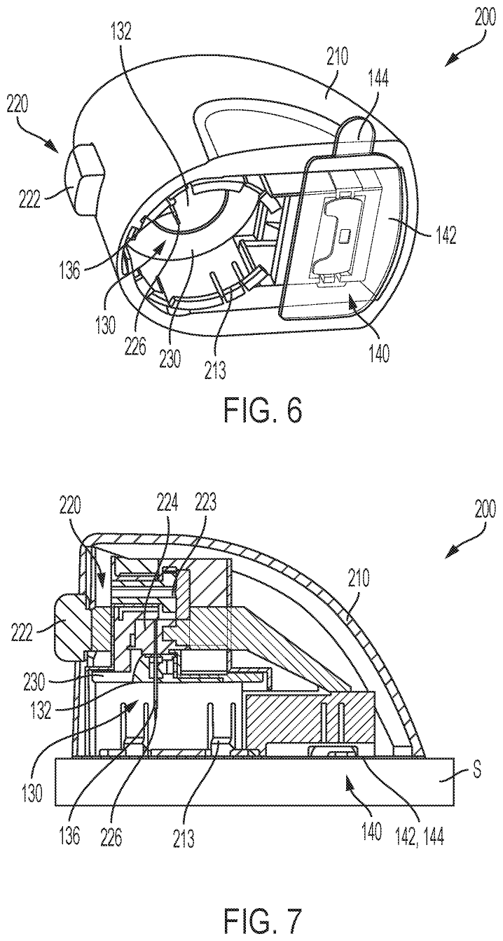

Referring next to FIGS. 6-13, a first exemplary applicator assembly 200 is provided for use with the drug infusion system 100 of FIGS. 1-5. The applicator assembly 200 may be supplied with the drug infusion system 100 and considered part of the drug infusion system 100. The applicator assembly 200 may be a multi-functional device configured to (1) selectively apply the infusion head 130 and/or the holder 140 to the patient's skin S and (2) insert the cannula 136 of the infusion head 130 into the patient's skin S.

The illustrative applicator assembly 200 includes a housing 210 configured to carry the infusion head 130 and the holder 140 in a predetermined arrangement. The predetermined arrangement may correspond to the patch configuration of FIG. 5, for example. The housing 210 may be sized to surround both the first adhesive pad 132 of the infusion head 130 and the second adhesive pad 142 of the holder 140, as shown in FIG. 6. In certain embodiments, the housing 210 may include one or more release features, such as opening 212 of FIG. 13 and/or an actuatable release mechanism (not shown), to facilitate separation of the infusion head 130 and/or the holder 140 from the housing 210. The opening 212 is described in more detail below in connection with FIG. 13. The housing 210 may also include one or more snap arms 213, which are described in more detail below in connection with FIG. 9.

The illustrative applicator assembly 200 also includes an inserter subassembly 220 configured to insert the cannula 136 of the infusion head 130 into the patient's skin S. The inserter subassembly 220 includes an actuator 222 (e.g., button), a rotary linkage 223, and an introducer hub 224 that carries an introducer needle 226. Although the illustrative inserter subassembly 220 controls both the insertion and retraction of the introducer hub 224 via the same actuator 222, it is also within the scope of the present disclosure to provide separate actuators for automatic and/or manual insertion and/or retraction of the introducer hub 224.

The illustrative applicator assembly 200 further includes an optional alignment guide 230 supported atop the infusion head 130 in the housing 210. The alignment guide 230 is shape-matched to the infusion head 130 to prevent lateral or rotary movement therebetween.

An exemplary method of using the applicator assembly 200 will now be described with continued reference to FIGS. 6-13. This method may be performed by the patient and/or the patient's caregiver, hereinafter a “user”. The applicator assembly 200 may have an ergonomic shape sized for receipt in the user's hand throughout this method.

In FIG. 6, the applicator assembly 200 is shown in a preparation state during a preparation step. During this preparation step, the user prepares the drug infusion system 100 for use in the desired wear configuration. To accommodate the tethered configuration (FIG. 4), the user may choose to apply only the infusion head 130 to the patient's skin S by ensuring that the first adhesive pad 132 of the infusion head 130 is exposed, while leaving the backing 144 on the second adhesive pad 142 of the holder 140, as shown in FIG. 6. To accommodate the patch configuration (FIG. 5), on the other hand, the user may choose to apply both the infusion head 130 and the holder 140 to the patient's skin S by ensuring that both the first and second adhesive pads 132, 142 are exposed, which may involve removing the backing 144 from the second adhesive pad 142. The infusion head 130 and the holder 140 and their first and second adhesive pads 132, 142 may be carried by the housing 210 in this preparation state, as shown in FIG. 6.

In FIG. 7, the applicator assembly 200 is shown in an application state during an application step. During this application step, the user positions the applicator assembly 200 in contact with the patient's skin S. If the user chooses not to attach the holder 140 to the skin S, the backing 144 will be retained to conceal the second adhesive pad 142 and prevent contact with the skin S, as shown in FIG. 7. If the user chooses to attach the holder 140 to the patient's skin S, on the other hand, the second adhesive pad 142 will be exposed to contact and adhere to the skin S. The cannula 136 of the infusion head 130 may be retracted into the housing 210 above the holder 140 in this application state to avoid premature contact with the patient's skin S, as shown in FIG. 7.

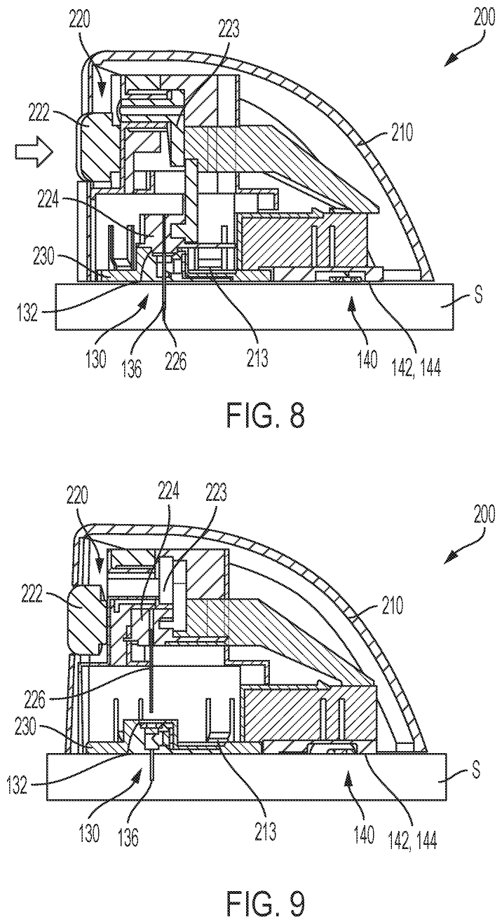

In FIG. 8, the applicator assembly 200 is shown in an insertion state during an insertion step. During this insertion step, the user presses the actuator 222 inward, which causes the rotary linkage 223 to rotate and drive the introducer hub 224, the alignment guide 230, and the infusion head 130 downward toward the patient's skin S past the snap arms 213 of the housing 210. This downward movement causes the introducer needle 226 to puncture the patient's skin S and allows the cannula 136 to enter the punctured site. The infusion head 130 moves from the retracted position of FIG. 7 into alignment with the holder 140 for mutual contact with the patient's skin S, and the exposed first adhesive pad 132 of the infusion head 130 contacts and adheres to the patient's skin S. Although the injection device of the illustrative infusion head 130 is a cannula 136, the injection device may take another form, such as a needle. In this case, the introducer hub 224 and the introducer needle 226 may be unnecessary.

In FIG. 9, the applicator assembly 200 is shown in a retraction state during a retraction step. During this retraction step, the rotary linkage 223 recoils and pulls the introducer hub 224 and the introducer needle 226 upward and out of the patient's skin S. As the introducer hub 224 moves upward, the snap arms 213 of the housing 210 hold the alignment guide 230 and the infusion head 130 downward. The adhered connection between the first adhesive pad 132 of the infusion head 130 and the patient's skin S may also keep the infusion head 130 downward, thereby allowing the cannula 136 of the infusion head 130 to remain implanted in the patient's skin S in the retraction state. The inserter subassembly 220, including the actuator 222, may be retained in this retraction state to prevent unnecessary reinsertion of the introducer needle 226.

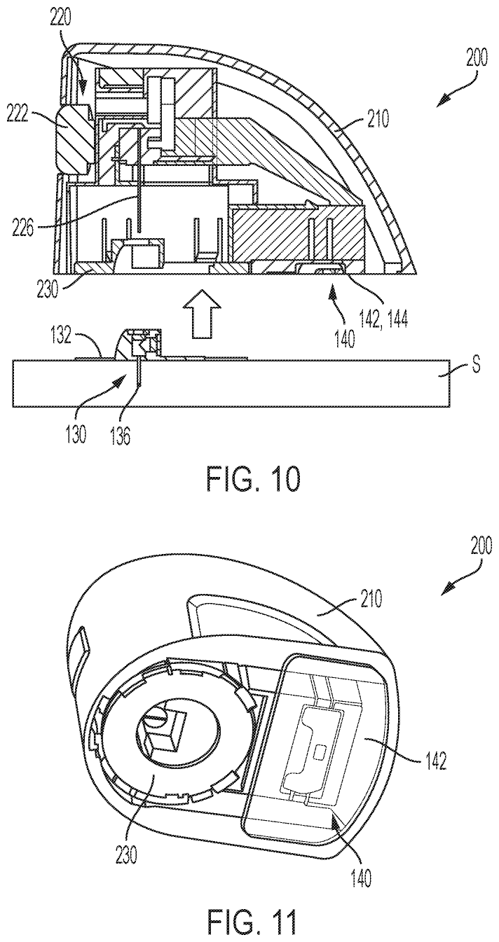

In FIG. 10, the applicator assembly 200 is shown in a separation state during a separation step. During this separation step, the user lifts the applicator assembly 200 off the patient's skin S, leaving behind the adhered infusion head 130 and/or the adhered holder 140. The adhesive strength of the first and second adhesive pads 132, 142, may be sufficient to release the adhered infusion head 130 and/or the adhered holder 140 from the housing 210 of the applicator assembly 200. In other embodiments, the user may use the release feature(s) of the housing 210, such as the opening 212 of FIG. 13, to facilitate such release. The opening 212 is described in more detail below in connection with FIG. 13. In the separation state of FIG. 10, the user has decided not to attach the holder 140 to the patient's skin S by continuing to conceal the second adhesive pad 142 with the backing 144. The lack of exposed adhesive between the holder 140 and the patient's skin S allows the housing 210 to remain inside the housing 210 and separated from the patient's skin S.

In FIG. 11, the applicator assembly 200 is shown in a second preparation state during a second preparation step. During this second preparation step, the user may prepare to adjust the drug infusion system 100 from the tethered configuration (FIG. 4) to the patch configuration (FIG. 5). The user may choose to apply the holder 140 to the patient's skin S (unless it was previously applied during the prior application step of FIG. 7) by removing the backing 144 (FIG. 6) from the second adhesive pad 142.

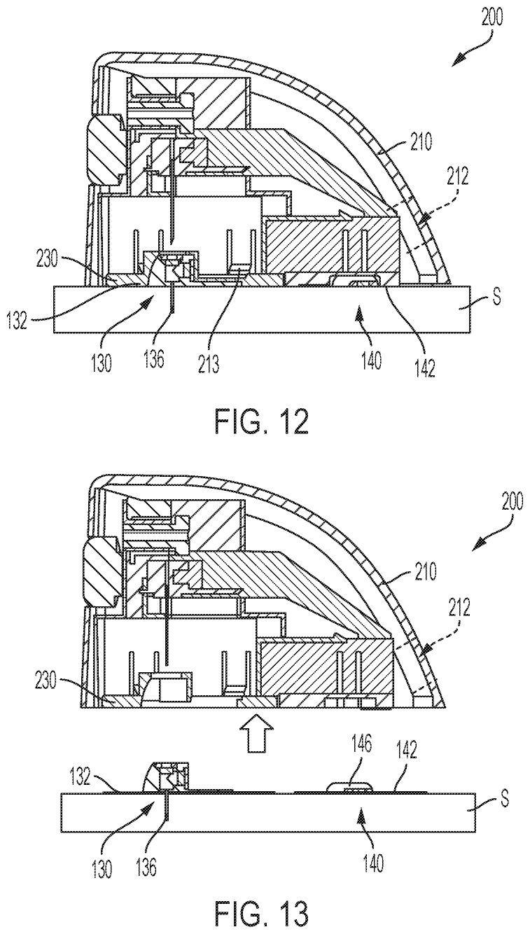

In FIG. 12, the applicator assembly 200 is shown in a second application state during a second application step. During this second application step, the user positions the applicator assembly 200 in contact with the patient's skin S while mating the alignment guide 230 with the inserted infusion head 130. The shape-matched conformity between the alignment guide 230 and the inserted infusion head 130 controls the location of the holder 140 relative to the inserted infusion head 130. With the second adhesive pad 142 now exposed, the holder 140 will adhere to the skin S at a predetermined location controlled by the alignment guide 230. This predetermined location of the holder 140 may be the same regardless of whether the holder 140 was applied together with the infusion head 130 during the first application step of FIG. 7 or after the infusion head 130 during the second application step of FIG. 12. Rather than using the integrated alignment guide 230, it is also within the scope of the present disclosure to use a separate tool (e.g., a separate spacer, a separate alignment plate) to control the location of the holder 140 relative to the inserted infusion head 130.

In FIG. 13, the applicator assembly 200 is shown in a second separation state during a second separation step. During this second separation step, the user lifts the applicator assembly 200 off the patient's skin S, leaving behind the newly adhered holder 140. The adhesive strength of the second adhesive pad 142 may be sufficient to release the adhered holder 140 from the housing 210 of the applicator assembly 200. In other embodiments, the user may use the release feature(s) of the housing 210, such as the opening 212 of FIG. 13, to facilitate such release. For example, the user may insert his or her finger into the opening 212 and press atop the holder 140 with a downward release force.

Once the infusion head 130 and the holder 140 have been applied to the patient's skin S, the user may adjust the drug infusion system 100 from the patch configuration (FIG. 5) to the tethered configuration (FIG. 4) by simply pulling and removing the holder 140 from the patient's skin S, without disturbing the inserted infusion head 130.

It is understood that the above method of using the applicator assembly 200 may be varied depending on the needs of the particular patient, such as by re-ordering and/or eliminating certain steps. For example, the second preparation step of FIG. 11, the second application step of FIG. 12, and the second separation step of FIG. 13 may be eliminated if the user applies the holder 140 during the first application step of FIG. 7, or if user only uses the drug infusion system 100 in the tethered configuration without the holder 140 (FIG. 4). Advantageously, the same applicator assembly 200 can be used throughout this customizable method, thereby minimizing manufacturing, storage, and purchase costs and maximizing flexibility for the user.

### 3. Second Applicator Assembly

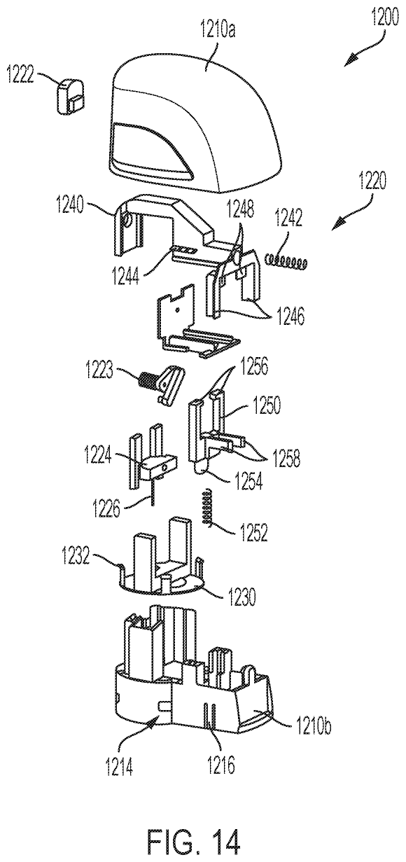

Referring next to FIG. 14, a second exemplary applicator assembly 1200 is provided for use with the drug infusion system 100 of FIGS. 1-5. The second applicator assembly 1200 may be similar to the first applicator assembly 200 of FIGS. 6 and 7, with like reference numerals indicating like elements, except as described below.

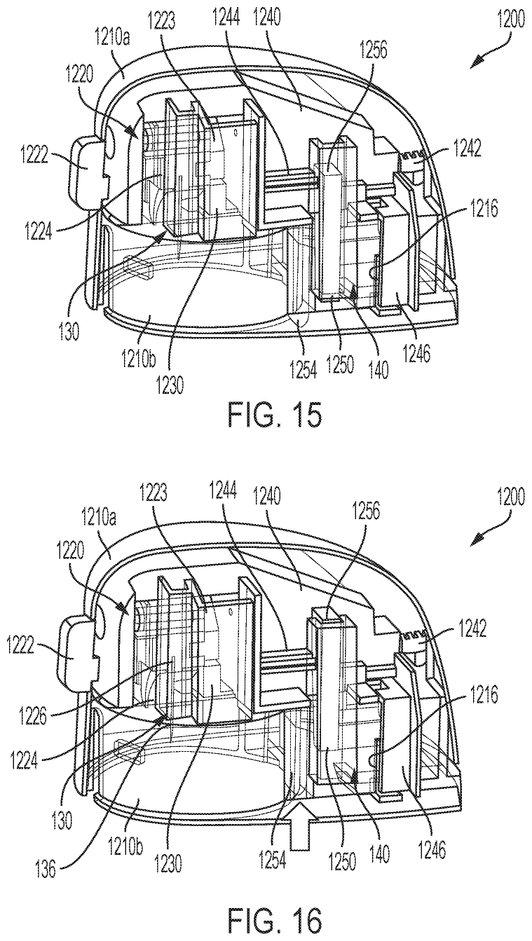

The second applicator assembly 1200 includes an upper housing 1210a and a lower housing 1210b configured to support the infusion head 130 and the holder 140 (FIG. 15). The second applicator assembly 1200 also includes an inserter subassembly 1220 having an actuator 1222, a rotary linkage 1223, and an introducer hub 1224 that carriers an introducer needle 1226. The second applicator assembly 1200 further includes an alignment guide 1230 having snap arms 1232. The lower housing 1210b includes windows 1214 that capture the snap arms 1232 of the alignment guide 1230 during insertion of the infusion head 130, similar to the snap arms 213 of FIG. 9. The lower housing 1210b also includes flexible detent beams 1216 that cooperate with the holder 140 (FIG. 15).

The illustrative applicator assembly 1200 also includes a carriage 1240 that prevents premature release of the holder 140 (FIG. 15). The carriage 1240 is coupled to the actuator 1222. The carriage 1240 includes a spring 1242 that biases the actuator 1222 outward, rails 1244, clamping arms 1246 that cooperate with the detent beams 1216 of the lower housing 1210b, and catches 1248.

The illustrative applicator assembly 1200 further includes a safety lock 1250 that prevents premature insertion of the infusion head 130 (FIG. 15). The safety lock 1250 includes a spring 1252 that biases the safety lock 1250 downward, a contact tab 1254 that cooperates with the patient's skin, locking tabs 1256 that cooperate with the rails 1244 of the carriage 1240, and snap arms 1258 that cooperate with the catches 1248 of the carriage 1240.

An exemplary method of using the second applicator assembly 1200 will now be described with reference to FIGS. 15-18.

In FIG. 15, the second applicator assembly 1200 is shown in a preparation state during a preparation step. During this preparation step, the safety lock 1250 is biased downward with the contact tab 1254 extending vertically beneath the housings 1210a, 1210b and the locking tabs 1256 positioned in horizontal alignment with the rails 1244 on the carriage 1240. This interaction between the locking tabs 1256 of the safety lock 1250 and the rails 1244 of the carriage 1240 prevents horizontal movement of the carriage 1240 toward the safety lock 1250. As a result, this interaction prevents premature triggering of the actuator 1222. Also, the carriage 1240 is biased rearward with the clamping arms 1246 positioned over the detent beams 1216 on the lower housing 1210b. This interaction between the clamping arms 1246 of the carriage 1240 and the detent beams 1216 of the lower housing 1210b forces the detent beams 1216 into engagement with the holder 140 and prevents the detent beams 1216 from flexing outward away from the holder 140. As a result, this interaction prevents premature release of the holder 140. This preparation state may also be referred to as a “fully locked” state, because both the actuator 1222 and the holder 140 are locked. This preparation state may have other features in common with the preparation state of FIG. 6.

In FIG. 16, the second applicator assembly 1200 is shown in an application state during an application step. During this application step, the user positions the applicator assembly 1200 in contact with the patient's skin, which causes the contact tab 1254 of the safety lock 1250 to move upward against the bias of the spring 1252. This upward movement of the safety lock 1250 frees the locking tabs 1256 of the safety lock 1250 from the rails 1244 of the carriage 1240, which allows triggering of the actuator 1222 and forward movement of the carriage 1240. However, the clamping arms 1246 of the carriage 1240 remain engaged with the detent beams 1216 of the lower housing 1210b to retain the holder 140. The upward movement of the safety lock 1250 also moves the snap arms 1258 of the safety lock 1250 into horizontal alignment with the catches 1248 of the carriage 1240 (FIG. 14). This application state may also be referred to as a “partially locked” state, because the actuator 1222 is unlocked and the holder 140 is locked. This application state may have other features in common with the application state of FIG. 7.

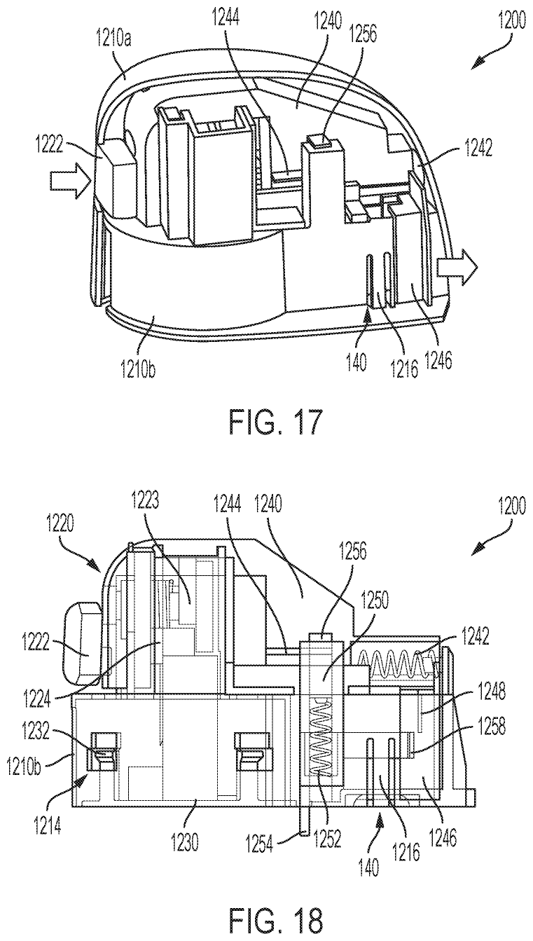

In FIG. 17, the second applicator assembly 1200 is shown in an insertion state during an insertion step. During this insertion step, the user presses the actuator 1222 forward, which causes forward movement of the carriage 1240 against the bias of the spring 1242. As has been described previously with respect to the insertion step of FIG. 8, and with reference once again to FIG. 16, this forward movement of the carriage releases the rotary linkage 1223, enabling it to rotate and drive the introducer hub 1224, the alignment guide 1230, and the infusion head 130 downward toward the patient's skin S past the snap arms 1213 of the housing 1210b. This downward movement causes the introducer needle 1226 to puncture the patient's skin S and allows the cannula 136 to enter the punctured site. The infusion head 130 moves from the retracted position of FIG. 15 into alignment with the holder 140 for mutual contact with the patient's skin S, and the infusion head 130 contacts and adheres to the patient's skin S. The forward movement of the carriage 1240 also frees the clamping arms 1246 of the carriage 1240 from the detent beams 1216 of the lower housing 1210b, thereby freeing the detent beams 1216. Returning to FIG. 17, although the detent beams 1216 are now free, the holder 140 remains gripped by the beams, as there is no force to cause it to be released. The forward movement of the carriage 1240 also moves the catches 1248 of the carriage 1240 past the snap arms 1258 of the safety lock 1250 (FIG. 14). The snap arms 1258 of the safety lock 1250 expand behind the catches 1248 of the carriage 1240, thereby preventing return or rearward movement of the carriage 1240 and the actuator 1222 to the initial position of FIG. 15. This insertion state may also be referred to as an “unlocked” state, because both the actuator 1222 and the holder 140 are unlocked. This insertion state may have other features in common with the insertion state of FIG. 8.

In FIG. 18, the second applicator assembly 1200 is shown in a separation state during a separation step. During this separation step, the user lifts the applicator assembly 200 off the patient's skin S. If the removable backing 144 (FIG. 1) has been removed from the holder 140, then the exposed adhesive 142 (FIG. 1) on the holder 140 adheres to the patient's skin S, and the act of pulling the second applicator assembly 1200 away from the skin S provides the force needed to release the holder 140 from the detent beams 1216. As the user continues to remove the second applicator assembly 1200 from the skin S, the spring 1252 returns the safety lock 1250 downward. The snap arms 1258 of the safety lock 1250 may drop beneath the catches 1248 of the carriage 1240, thereby allowing return or rearward movement of the carriage 1240 and the actuator 1222. As the carriage 1240 moves rearward, the rails 1244 of the carriage 1240 may slide beneath the locking tabs 1256 of the safety lock 1250. When the carriage 1240 passes the locking tabs 1256 and resumes its initial position (FIG. 15), the safety lock 1250 may continue downward until the locking tabs 1256 reengage the rails 1244 and resume preventing premature triggering of the actuator 1222. Also, the clamping arms 1246 of the carriage 1240 may reengage the detent beams 1216 on the lower housing 1210b and resume preventing premature release of the holder 140, unless it was previously released. This separation state may also be referred to as a “relocked” state, because both the actuator 1222 and the holder 140, if it remains, are locked. This separation state may have other features in common with the separation state of FIG. 10.

The operation of the carriage 1240 and the safety lock 1250 may vary. For example, rather than unlocking the safety lock 1250 when the contact tab 1254 contacts the patient's skin, the safety lock 1250 may be unlocked with a separate button or slide mechanism, with a dual-function actuator having a first unlocking movement (e.g., slide) and a second inserting movement (e.g., press), or with various other mechanisms. In another example, the user may maintain pressure on the actuator 1222 to maintain the carriage 1240 in the forward position rather than relying on the snap arms 1258 to lock the carriage 1240 and the actuator 1222 in the forward position.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An applicator assembly configured for use with a drug infusion system including a drug delivery device, the drug infusion system having a tethered configuration and a patch configuration, the applicator assembly comprising:
an infusion head having a first adhesive pad and an injection device;
a holder having a second adhesive pad, the holder configured to support the drug delivery device;
a housing configured to carry the infusion head and the holder; and
an actuator coupled to the housing and configured to move the injection device from the housing into a patient's skin.

2. The applicator assembly of claim 1, wherein the applicator assembly has a preparation state in which:
for the tethered configuration, the first adhesive pad of the infusion head is exposed to the patient's skin and the second adhesive pad of the holder is concealed from the patient's skin; and
for the patch configuration, the first adhesive pad of the infusion head is exposed to the patient's skin and the second adhesive pad of the holder is exposed to the patient's skin.

3. The applicator assembly of claim 1 , wherein the holder further includes a removeable backing that conceals the second adhesive pad in the tethered configuration.

4. The applicator assembly of claim 1, wherein the applicator assembly has:
an application state in which the holder contacts the patient's skin and the injection device of the infusion head is retracted into the housing above the holder; and
an insertion state in which the infusion head is aligned with the holder for mutual contact with the patient's skin.

5. The applicator assembly of claim 1, wherein the injection device is a cannula and the applicator assembly further comprises an introducer hub with an introducer needle that punctures the patient's skin for receipt of the cannula in the insertion state, wherein the applicator assembly has a retraction state in which the introducer needle retracts into the housing and the cannula remains in the patient's skin.

6. The applicator assembly of claim 1, further comprising an alignment guide positioned in the housing, wherein the applicator assembly has:
a second preparation state in which the infusion head is adhered to the patient's skin and the holder remains positioned in the housing; and
a second application state in which the alignment guide mates with the infusion head to adhere the holder to the patient's skin at a predetermined location relative to the infusion head.

7. The applicator assembly of claim 1, wherein the housing surrounds the first adhesive pad of the infusion head and the second adhesive pad of the holder.

8. The applicator assembly of claim 1, further comprising a safety lock having:
a locked state that prevents movement of the actuator; and
an unlocked state that permits movement of the actuator to move the injection device.

9. The applicator assembly of claim 8, wherein the safety lock includes a contact tab that extends beneath the housing in the locked state and retracts into the housing upon contact with the patient's skin in the unlocked state.

10. The applicator assembly of claim 1, further comprising a carriage coupled to the actuator and having:
a first state in which the carriage cooperates with the holder to prevent release of the holder from the housing; and
a second state in which the carriage disengages the holder to permit release of the holder from the housing.

11. The applicator assembly of claim 10, further comprising a safety lock having:
a locked state that locks the carriage in the first state; and
an unlocked state that permits movement of the carriage to the second state.

12. A drug infusion system comprising:
a drug delivery device;
an infusion head having an injection device;
a holder configured to support the drug delivery device; and
an applicator assembly comprising:
a housing configured to carry the infusion head and the holder; and
an actuator coupled to the housing and configured to move the injection device from the housing into a patient's skin;
wherein the drug infusion system has:
a tethered configuration in which the applicator assembly positions the infusion head in adhesive communication with the patient's skin while preventing adhesive communication between the holder and the patient's skin; and
a patch configuration in which the applicator assembly positions the infusion head and the holder in adhesive communication with the patient's skin.

13. The drug infusion system of claim 12, further comprising a tubing set positioned in fluid communication between the drug delivery device and the infusion head in the tethered configuration.

14. The drug infusion system of claim 13, wherein:
the drug delivery device includes a needle port;
the infusion head includes a septum port; and
the tubing set includes a septum port at one end and a needle port at the other end;
wherein:
in the tethered configuration, the needle port of the drug delivery device communicates with the septum port of the tubing set, and the needle port of the tubing set communicates with the septum port of the infusion head; and
in the patch configuration, the needle port of the drug delivery device communicates with the septum port of the infusion head without the tubing set.

15. The drug infusion system of claim 13, wherein:
the drug delivery device includes a male connector;
the infusion head includes a female connector; and
the tubing set includes a female connector at one end and a male connector at the other end;
wherein:
in the tethered configuration, the male connector of the drug delivery device couples to the female connector of the tubing set, and the male connector of the tubing set couples to the female connector of the infusion head; and
in the patch configuration, the male connector of the drug delivery device couples to the female connector of the infusion head without the tubing set.

16. The drug infusion system of claim 13, wherein the tubing set is stored in a closed-loop in the patch configuration with one end of the tubing set coupled to the other end of the tubing set.

17. The drug infusion system of claim 12, wherein the applicator assembly further includes an alignment guide configured to adjust the drug infusion system from the tethered configuration to the patch configuration, the applicator assembly mating the alignment guide with the infusion head while positioning the holder in adhesive communication with the patient's skin.

18. A method of using a drug infusion system comprising the steps of:
obtaining an applicator assembly comprising a housing, an infusion head having a first adhesive pad and an injection device, and a holder having a second adhesive pad;
preparing the applicator assembly by selectively concealing or exposing the second adhesive pad of the holder;
applying the applicator assembly to a patient's skin, the holder adhering to the patient's skin during the applying step if the second adhesive pad is exposed during the preparing step;
actuating the applicator assembly to adhere the first adhesive pad of the infusion head to the patient's skin and insert the injection device into the patient's skin; and
separating the applicator assembly from the patient's skin, the holder separating from the patient's skin during the separating step if the second adhesive pad is concealed during the preparing step.

19. The method of claim 18, wherein, after separating the holder from the patient's skin during the separating step, the method further comprises the step of:
adjusting the applicator assembly by exposing the second adhesive pad of the holder; and
reapplying the applicator assembly to the patient's skin using an alignment guide to adhere the holder to the patient's skin at a predetermined location relative to the infusion head in the patient's skin.

20. The method of claim 18, further comprising the steps of:
wearing the drug infusion system in a tethered configuration by placing a drug delivery device in fluid communication with the infusion head via an intermediate tubing set; or
wearing the drug infusion system in a patch configuration by coupling the drug delivery device to the holder and placing the drug delivery device in fluid communication with the infusion head without the intermediate tubing set.
